(19) United States
(12) Patent Application Publication
(10) Pub. No.: US 2024/0423585 A1
(43) Pub. Date: Dec. 26, 2024

(54) INTRACAVITARY INSERTION TYPE ULTRASOUND PROBE

(71) Applicant: FUJIFILM Healthcare Corporation, Kashiwa-shi (JP)

(72) Inventor: Takahide Terada, Tokyo (JP)

(21) Appl. No.: 18/670,153

(22) Filed: May 21, 2024

(30) Foreign Application Priority Data
Jun. 22, 2023 (JP) 2023-102767

(51) Int. Cl. A61B 8/12; A61B 8/00

(52) U.S. Cl. CPC A61B 8/12; A61B 8/4488; A61B 8/4494

(57) ABSTRACT

An image quality of an ultrasound image formed by using an intracavitary insertion type ultrasound probe can be improved, and a degree of freedom in design can be increased. M electronic circuits IC1 to IC5 are connected to a transducer array 80. Each of the electronic circuits IC1 to IC5 has a multiplexer MU and i receivers R1 to R4. The multiplexer MU includes (i×j+k) (where j is an integer of one or more and k is an integer of one or more and less than i) input terminals and i output terminals. A wiring circuit 200 including the M electronic circuits IC1 to IC5 connects i signal lines L1 to L4 to i transducers constituting a reception opening without overlapping connection.

### CROSS-REFERENCE TO RELATED APPLICATION

The present application claims priority from Japanese Patent Application No. 2023-102767 filed on Jun. 22, 2023, the content of which is hereby incorporated by reference into this application.

### BACKGROUND OF THE INVENTION

### 1. Field of the Invention

The present disclosure relates to an intracavitary insertion type ultrasound probe, and more particularly, to a technique of connecting a plurality of signal lines to a transducer array.

### 2. Description of the Related Art

As the intracavitary insertion type ultrasound probe, a small diameter probe such as an intravascular ultrasound (IVUS) probe is known. Such a probe is also referred to as a catheter type ultrasound probe.

In an electronic scanning IVUS probe in the related art, an annular flexible printed circuit (FPC) substrate is disposed in a tip part of the electronic scanning IVUS probe. An annular transducer array and a plurality of electronic circuits connected to the transducer array are provided on an inner side of the annular FPC substrate.

JP2022-544560A discloses an electronic scanning IVUS probe. A plurality of ICs are mounted on an FPC substrate provided in a tip part of the probe. Each IC has a 16-to-1multiplexer that connects one transducer selected from 16 transducers to one signal line. One reception signal is acquired per one transmission/reception. JP2013-165865A also discloses an electronic scanning IVUS probe.

JP2022-544560A and JP2013-165865A do not disclose a mechanism for simultaneously acquiring a plurality of reception signals and transmitting the plurality of reception signals to an ultrasound diagnostic apparatus main body. In particular, JP2022-544560A and JP2013-165865A do not disclose an IC provided with input terminals whose number is not an integer multiple of a simultaneous reception number.

### SUMMARY OF THE INVENTION

In order to improve an image quality of an ultrasound image generated by using the intracavitary insertion type ultrasound probe, the plurality of reception signals are desired to be simultaneously acquired. On the premise of the above, a degree of freedom in designing the intracavitary insertion type ultrasound probe is desired to be increased.

An object of the present disclosure is to provide an intracavitary insertion type ultrasound probe capable of improving an image quality of an ultrasound image. Alternatively, an object of the present disclosure is to increase a degree of freedom in design regarding the intracavitary insertion type ultrasound probe.

An intracavitary insertion type ultrasound probe according to the present disclosure comprises an annular transducer array, i (where i is an integer of two or more) signal lines, and a wiring circuit including a plurality of multiplexers that are disposed side by side in parallel between the transducer array and the i signal lines, in which each of the multiplexers has (i×j +k) (where j is an integer of one or more and k is an integer of one or more and less than i) input terminals to which a plurality of reception signals from the transducer array are input and i output terminals connected to the i signal lines, and the wiring circuit connects, in a reception beam scanning process in which a plurality of reception openings are sequentially set to the transducer array, the i signal lines to i transducers constituting each reception opening without overlapping connection.

According to the present disclosure, it is possible to improve the image quality of the ultrasound image. Alternatively, according to the present disclosure, it is possible to increase the degree of freedom in design of the intracavitary insertion type ultrasound probe.

### BRIEF DESCRIPTION OF THE DRAWINGS

FIG. 1 is a schematic view of an electronic scanning IVUS probe according to an embodiment.

FIG. 2 is a cross-sectional view of a tip part shown in FIG. 1.

FIG. 3 is a diagram showing a development state of an assembly.

FIG. 4 is a diagram showing an implemented state of the assembly.

FIG. 5 is a block diagram showing a configuration of an electronic circuit according to a first example.

FIG. 6 is a circuit diagram showing an entirety of a reception system according to the first example.

FIG. 7 is a circuit diagram showing a part of the reception system according to the first example.

FIG. 8 is a table showing a transmission element group and a reception element group.

FIG. 9 is a diagram showing a transfer method of control data.

FIG. 10 is a diagram showing an FPC substrate for wiring according to the first example.

FIG. 11 is a diagram showing a configuration of an electronic circuit according to a second example.

FIG. 12 is a circuit diagram showing an entirety of a reception system according to the second example.

FIG. 13 is a circuit diagram showing a part of the reception system according to the second example.

FIG. 14 is a circuit diagram showing an entirety of a reception system according to a third example.

FIG. 15 is a circuit diagram showing a part of the reception system according to the third example.

FIG. 16 is a diagram showing a configuration of an electronic circuit according to a modification example.

FIG. 17 is a circuit diagram showing an entirety of a reception system according to a fourth example.

FIG. 18 is a circuit diagram showing a part of the reception system according to the fourth example.

### DESCRIPTION OF THE PREFERRED EMBODIMENTS

An embodiment will now be described with reference to accompanying drawings.

### (1) Outline of Embodiment

An intracavitary insertion type ultrasound probe according to the embodiment includes an annular transducer array, i (where i is an integer of two or more) signal lines, and a wiring circuit having a plurality of multiplexers. The plurality of multiplexers are disposed in parallel between the transducer array and i signal lines. Each multiplexer includes (i×j+k) input terminals (where j is an integer of one or more and k is an integer of one or more and less than i) to which a plurality of reception signals from the transducer array are input and i output terminals connected to the i signal lines. In a reception beam scanning process in which a plurality of reception openings are sequentially set for the transducer array, the wiring circuit connects the i signal lines to i transducers constituting each reception opening without overlapping connection.

The number of signal lines i corresponds to the number of reception signals that can be transferred in parallel at the same time, that is, a simultaneous reception number. The number of transducers connected to each multiplexer may be set to an integer multiple of the simultaneous reception number i in a common configuration. However, such a common configuration may not be employed due to design reasons. In such a case, the above configuration according to the embodiment effectively functions.

In the above configuration, the number of input terminals (i×j+k) of each multiplexer is not the integer multiple of the number of signal lines i. In a case where the number of input terminals (i×j+k) is divided by the number of signal lines i, a remainder k is generated. On the premise of such a configuration, the wiring circuit connects the i signal lines to the i transducers constituting the reception opening without overlapping connection. Here, the overlapping connection is generally a state in which a plurality of transducers are simultaneously connected to one signal line, or a state in which a plurality of signal lines are simultaneously connected to one transducer. The wiring circuit is to perform wiring in accordance with the above remainder k, that is, to avoid the occurrence of the overlapping connection and realize one-to-one connection.

With the above configuration, it is possible to obtain a plurality of reception signals at the same time in one transmission and reception, and thus improve an image quality of an ultrasound image. In addition, it is possible to independently define the number of transducers, the number of multiplexers, the number of input terminals, and the number of signal lines, and thus increase a degree of freedom in design.

In the embodiment, each multiplexer includes (i×j+k) connection paths for circularly connecting the i output terminals one-to-one, from a head input terminal, which is a first input terminal, to a last input terminal, which is an (i×j+k)-th input terminal.

In a certain example, each multiplexer has only the (i×j+k) connection paths. In another example, each multiplexer has ((i×j+k+(i−1) connection paths. The added (i−1) connection paths are for avoiding the overlapping connection in a second situation described below. A multiplexer having (i×j+k)×i connection paths can be used as each multiplexer, but in this case, the circuit scale of the multiplexer is increased. In the embodiment, the number of connection paths is equal to or larger than (i×j+k) and less than (i×j+k)×i in each multiplexer.

In the embodiment, the plurality of multiplexers are M (where M is an integer of two or more) multiplexers. In a first situation in which the reception opening is set across an x-th (where x is 1, 2, . . . , and M−1) multiplexer and an (x+1)-th multiplexer adjacent to each other in an electronic scanning direction, in a case where a y-th (where y is an integer of 1 or more and i or less) signal line is connected to a last input terminal of the x-th multiplexer, the wiring circuit connects a (y+1)-th (where first in case of y=i) signal line to a head input terminal of the (x+1)-th multiplexer.

With the above configuration, in electronic scanning of the reception opening from a first multiplexer to an M-th multiplexer, the overlapping connection can be naturally avoided. In a configuration example described below, the multiplexer closest to one side of the FPC substrate is referred to as the first multiplexer, and the multiplexer closest to the other side of the FPC substrate is referred to as the M-th multiplexer. However, in the M multiplexers disposed annularly, any multiplexer may be defined as the first multiplexer.

In the embodiment, the (i×j+k) input terminals in the M-th multiplexer include use portions and non-use portions disposed side by side in the electronic scanning direction. In the second situation in which the reception opening is set across the M-th multiplexer and the first multiplexer, in a case where a z-th (where z is an integer of 1 or more and i or less) signal line is connected to a last input terminal of the use portion in the M-th multiplexer, the wiring circuit connects a (z+1)-th (where first in case of z=i) signal line to a head input terminal of the first multiplexer.

With the above configuration, even in a case where a part of an input terminal group of the M-th multiplexer is the non-use portion, the occurrence of the overlapping connection can be avoided in the second situation described above. With the electronic scanning of the reception opening, a connection relationship between the i signal lines and the i transducers may be reset to an initial connection state or a steady connection state at a point in time at which the second situation ends (that is, at a point in time at which the entire reception opening is within a responsible range of the first multiplexer).

In the embodiment, the plurality of multiplexers include at least one first multiplexer and at least one second multiplexer. In at least one first multiplexer, all of the (i×j+k) input terminals are used. In at least one second multiplexer, the (i×j+k) input terminals include the use portion and the non-use portion. The use portion is configured of a plurality of input terminals, and the non-use portion is configured of one or a plurality of input terminals. In general, the non-use portion is set on a downstream side of the use portion in the electronic scanning direction. The first multiplexer is an all-input-terminal use type multiplexer, and the second multiplexer is a partial-input-terminal non-use type multiplexer.

In a situation in which the reception opening is set across between the first multiplexer and a next multiplexer that follows the first multiplexer, in a case where the y-th signal line is connected to a last input terminal of the first multiplexer, the wiring circuit connects a (y+1)-th (where first in case of y=i) signal line to a head input terminal of the next multiplexer. The next multiplexer may be a head multiplexer.

On the other hand, in a situation in which the reception opening is set across between the second multiplexer and a next multiplexer that follows the second multiplexer, in a case where a z-th signal line is connected to a last input terminal of the use portion in the second multiplexer, the wiring circuit connects a (z+1)-th (where first in case of z=i) signal line to a head input terminal of the next multiplexer. The next multiplexer may be a head multiplexer. The plurality of multiplexers constituting the wiring circuit may include a plurality of second multiplexers.

The intracavitary insertion type ultrasound probe according to the embodiment further includes M electronic circuits provided between the transducer array and the i signal lines. Each electronic circuit includes a multiplexer and i receivers provided between the i output terminals of the multiplexer and the i signal lines.

Each of the individual receivers is, for example, an amplifier. With amplification of the reception signal in each of the individual receivers, the image quality of the ultrasound image is improved. A transmission circuit may be provided in each electronic circuit. The transmission circuit may be configured of (i×j+k) transmitters connected to (i×j+k) transducers. In the embodiment, the plurality of electronic circuits have the same configuration. Accordingly, it is possible to reduce a probe manufacturing cost.

The intracavitary insertion type ultrasound probe according to the embodiment further includes a circuit board having a wiring pattern in which the i signal lines are connected to the plurality of electronic circuits. In the embodiment, each electronic circuit has i internal signal lines drawn from the i receivers. The i internal signal lines are connected to the above wiring pattern. A specific internal signal line among i internal signal lines has two connection points. A portion between the two connection points in the specific internal signal line functions as a jumper (short-circuit line) connecting two signal lines separated from each other in the wiring pattern. With this configuration, it is possible to easily avoid the cross wiring, and thus it is possible to use a circuit board having a single wiring pattern layer. The specific internal signal line may have three or more connection points. In that case, two or more portions between the connection points are generated. The portions between the respective connection points may function as the jumper.

In the embodiment, the wiring circuit includes a sub-multiplexer provided between the i output terminals of the first multiplexer and the i signal lines. The M-th multiplexer, the first multiplexer, and the sub-multiplexer connect the i signal lines to the i transducers constituting the reception opening without overlapping connection in the second situation.

Depending on a combination of the number of transducers, the number of signal lines, the number of multiplexers, and the number of input terminals, the overlapping connection may occur in the second situation. With the use of the sub-multiplexer, the degree of freedom in wiring is increased. Therefore, it is possible to reliably avoid the overlapping connection. In a case where the configurations of the plurality of electronic circuits are made the same, a plurality of sub-multiplexers corresponding to the plurality of multiplexers are provided. However, the sub-multiplexers other than a first sub-multiplexer do not function. Each sub-multiplexer may be configured of a plurality of switch circuits.

In the embodiment, the wiring circuit includes an additional connection path group consisting of i−1 additional connection paths added to the first multiplexer. The M-th multiplexer and the first multiplexer connect the i signal lines to the i transducers constituting the reception opening without overlapping connection in the second situation. As described above, depending on a combination of the number of transducers, the number of signal lines, the number of multiplexers, and the number of input terminals, the overlapping connection may occur in the second situation. The additional connection path group effectively functions in such a case. In a case where the configurations of the plurality of electronic circuits are made the same, the additional connection path group is provided in each multiplexer. However, in the multiplexers other than the first multiplexer, the additional connection path group does not function. The number of additional connection paths exceeding (i−1) additional connection paths may be provided in the first multiplexer. As already described, in the embodiment, the number of connection paths is equal to or larger than (i×j+k) and less than (i×j+k)×i in each multiplexer.

### (2) Details of Embodiment

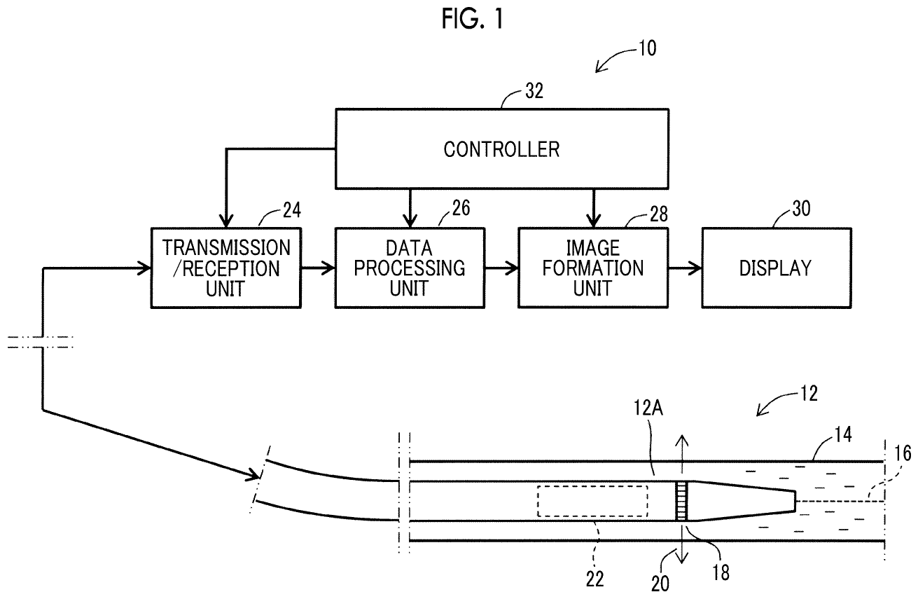

FIG. 1 shows an ultrasound diagnostic apparatus according to the embodiment. The ultrasound diagnostic apparatus is used in an ultrasound examination of a living body (person). The ultrasound diagnostic apparatus includes an apparatus main body 10 and an intracavitary insertion type ultrasound probe (hereinafter referred to as probe) 12. Specifically, the probe 12 is an electronic scanning IVUS probe to be inserted into a blood vessel 14. A configuration described below may be applied to another probe (for example, ultrasonic endoscope for bronchus).

The probe 12 is a long member having flexibility. An outer diameter of the probe 12 is, for example, in a range of 1 to 3 mm. The outer diameter of the probe 12 may be in the range thereof or less or in the range thereof or more.

A tip part 12A of the probe 12 has a transducer array 18 having an annular form. The transducer array 18 is configured of, for example, several tens or several hundreds of transducers disposed annularly. A transmission opening and the reception opening are set on the transducer array 18. An ultrasound wave is emitted from the transmission opening, and a reflected wave is received by the reception opening. An ultrasound beam (transmission/reception integrated beam) 20 is radially scanned by the electronic scanning of the openings.

An electronic circuit group 22 is provided in the tip part 12A. The electronic circuit group 22 is electrically connected to the transducer array 18. The transmission opening and the reception opening are electronically scanned by an action of the electronic circuit group 22. The electronic circuit group 22 is configured of the plurality of electronic circuits. The number of electronic circuits is, for example, in a range of 4 to 8, and may be 5 or 6. Each electronic circuit is configured of, for example, an application specific integrated circuit (ASIC).

The probe 12 has a hollow passage formed along a central axis thereof. A guide wire 16 is inserted into the passage. More accurately, the probe 12 advances in the blood vessel 14 along the guide wire 16 that is already disposed in the blood vessel 14.

The transmission/reception unit 24 is an electronic circuit that functions as a transmission control circuit and also functions as a reception beam former. Transmission/reception control data is transmitted from the transmission/reception unit 24 to the electronic circuit group 22. The electronic circuit group 22 generates a plurality of transmission signals during the transmission. The transmission signals are transmitted to the transducer array 18. With the transmission, a transmission beam is formed by the transducer array 18.

During the reception, the plurality of reception signals output in parallel from the transducer array 18 are transmitted to the transmission/reception unit 24 via the electronic circuit group 22. In the transmission/reception unit 24, coherent addition is applied to the plurality of reception signals. With the coherent addition, reception beam data is generated. The electronic circuit group 22 may perform sub-beam forming for reception.

Although an interface unit is provided between the probe 12 and the transmission/reception unit 24, an illustration thereof is omitted in FIG. 1. A plurality of pieces of reception beam data are sequentially transmitted from the transmission/reception unit 24 to an image formation unit 28 via a data processing unit 26 as the ultrasound beam is radially scanned.

The data processing unit 26 includes a detection circuit, a filter circuit, a logarithmic conversion circuit, and the like. The image formation unit 28 is configured of a digital scan converter (DSC). A tomographic image is formed from the plurality of pieces of reception beam data by the DSC. The tomographic image is displayed on a display 30. The tomographic image is, for example, a motion picture image showing a transverse cross section of the blood vessel 14. A motion picture image representing a longitudinal cross section of the blood vessel may be displayed on the display 30. In that case, a plurality of pieces of transverse cross section data may be connected in a blood vessel central axis direction to generate the tomographic image representing the longitudinal cross section of the blood vessel. A three-dimensional image may be displayed on the display 30.

A controller 32 has a central processing unit (CPU) that executes a program. The controller 32 controls an operation of each component in the ultrasound diagnostic apparatus. The display 30 is configured of an organic EL display device, a liquid crystal display (LCD), or the like. An operation panel (not shown) is connected to the controller 32.

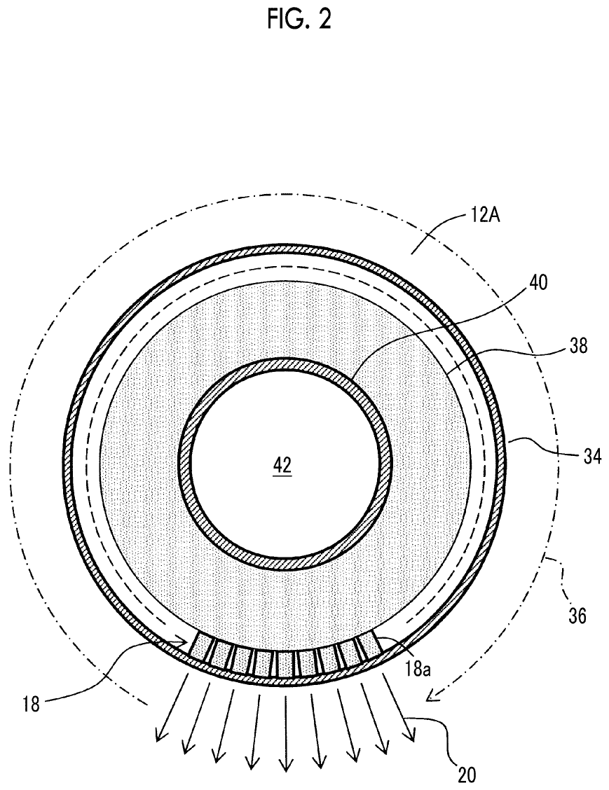

FIG. 2 shows a cross section of the probe according to the embodiment. Specifically, FIG. 2 shows a schematic view of a transverse cross section of the tip part 12A. An FPC substrate 34 is a film member for wiring having flexibility. The FPC substrate 34 has an annular form. The transducer array 18 is provided on an inner side of the FPC substrate 34. The transducer array 18 is configured of a plurality of transducers 18a disposed annularly. The ultrasound wave transmitted from each transducer 18a is transmitted through the FPC substrate 34 and then radiated into the living body. A reflected wave from the inside of the living body is transmitted through the FPC substrate 34 and then received by each transducer 18a. The ultrasound beam 20 is radially scanned as shown in the drawing (refer to reference numeral 36).

A cylindrical backing 38 is provided on an inner side of the transducer array 18. The backing 38 is a member that attenuates the ultrasound wave radiated rearward from each transducer 18a. A pipe 40 is provided on an inner side of the backing 38. An inside of the pipe 40 is a passage 42 into which the guide wire is inserted.

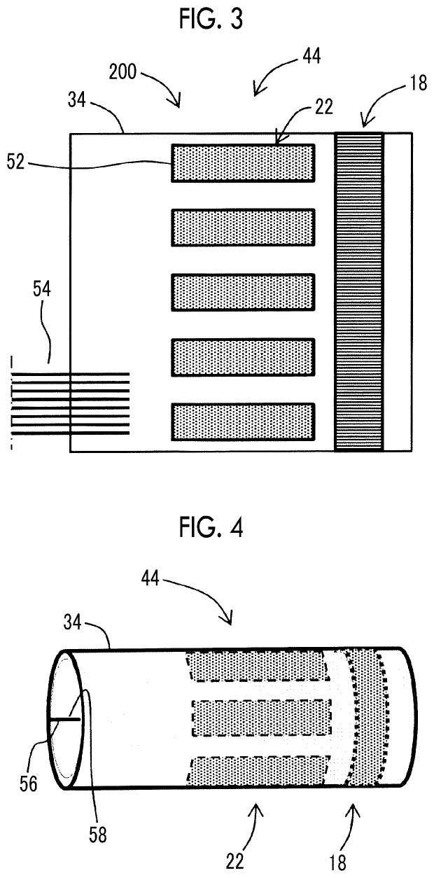

FIG. 3 shows an assembly 44 in a development state. The assembly 44 is disposed in the tip part of the probe. The assembly 44 is configured of the FPC substrate 34, the transducer array 18, the electronic circuit group 22, and the like. The electronic circuit group 22 is configured of a plurality of electronic circuits 52. A reference numeral 54 indicates a signal line group provided between the tip part of the probe and the ultrasound diagnostic apparatus main body. A wiring circuit 200 connects, during reception, a plurality of signal lines to a plurality of transducers constituting the reception opening without overlapping connection, that is, one-to-one. The wiring circuit 200 includes a plurality of multiplexers described below.

FIG. 4 shows the assembly 44 in an implemented state. The FPC substrate 34 is rolled and has a tubular form. In that state, one side 56 and the other side 58 in the FPC substrate 34 are close to or connected to each other. The transducer array 18 and the electronic circuit group 22 are provided on an inner side of the FPC substrate 34. For example, a direction from one side 56 to the other side 58 is the electronic scanning direction. Among M (where M is an integer of two or more) electronic circuits 52 constituting the electronic circuit group 22, the electronic circuit closest to one side 56 is a first electronic circuit 52, and the electronic circuit closest to the other side 58 is an M-th electronic circuit 52.

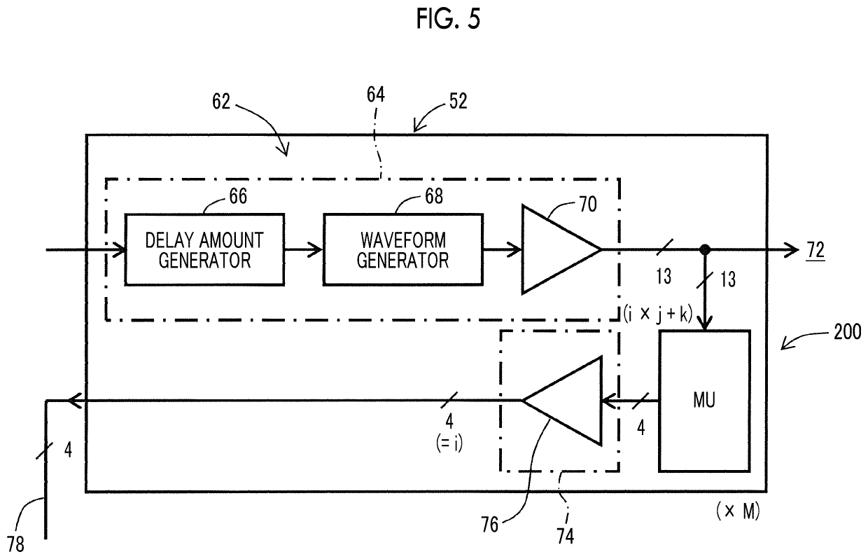

FIG. 5 shows the electronic circuit 52 according to a first example. The electronic circuit 52 is an integrated circuit and is specifically an ASIC. The electronic circuit 52 is connected to a specific transducer group 72 in the transducer array. The transducer group 72 consists of the (i×j+k) transducers. The transducer group connected to the M-th electronic circuit is configured of the number of transducers equal to or less than (i×j+k) .

Here, i corresponds to the number of signal lines for reception signal transmission. For example, i=4. In that case, four reception signals are simultaneously transmitted in parallel. j is an integer of one or more. k is an integer of one or more and less than i. In a case where the number of the transducers, which constitute the transducer group 72, (i×j+k) is divided by the number of the signal lines i, the remainder k is generated. For example, j is three and k is one. In the illustrated configuration example, (i×j+k)=13.

The electronic circuit 52 includes a transmission circuit 62, a reception circuit 74, and a multiplexer MU. The transmission circuit 62 is configured of, for example, (i×j+k) transmitters 64. Each transmitter 64 includes a delay amount generator 66, a waveform generator 68, and an amplifier 70. The delay amount generator 66 sets a transmission delay amount (delay time). The waveform generator 68 generates the transmission signal. The transmission signal subjected to delay processing is amplified by the amplifier 70. The transmission signal after amplification is output to the transducer corresponding to the transmission signal. In practice, (i ×j+k) transmission signals are output in parallel to the transducer group 72. With the above, the transmission beam is formed.

The multiplexer MU has the (i×j+k) input terminals and the i output terminals. In addition, the multiplexer MU has a connection path group consisting of the (i×j+k) connection paths connecting the (i×j+k) input terminals and the i output terminals. A maximum of i connection paths are simultaneously selected from the connection path group. The multiplexer MU has a large number of switches for path selection. A multiplexer that can sufficiently withstand a transmission voltage is used as the multiplexer MU.

The reception circuit 74 is configured of i receivers 76. Each of the individual receivers 76 is specifically a preamplifier. In a case where i reception signals are output in parallel from the multiplexer MU, all of i reception signals are amplified, and i amplified reception signals are transmitted to i signal lines 78. The i signal lines 78 are used for simultaneously and in parallel transmitting i reception signals to the ultrasound diagnostic apparatus main body.

During the transmission, each unused transmitter 64 is turned off, and in the multiplexer MU, all connections between the (i×j+k) input terminals and the i output terminals are cut off. During the reception, the transmission circuit 62 is turned off, and each unused receiver 76 is also turned off. In the multiplexer MU, a connection path corresponding to each receiver 76 used at each of individual points in time is selected from the connection path group.

The M electronic circuits 52 are disposed in the tip part of the probe. The M electronic circuits 52 include M multiplexers MU. In the first example, the wiring circuit 200 is configured by the M multiplexers MU.

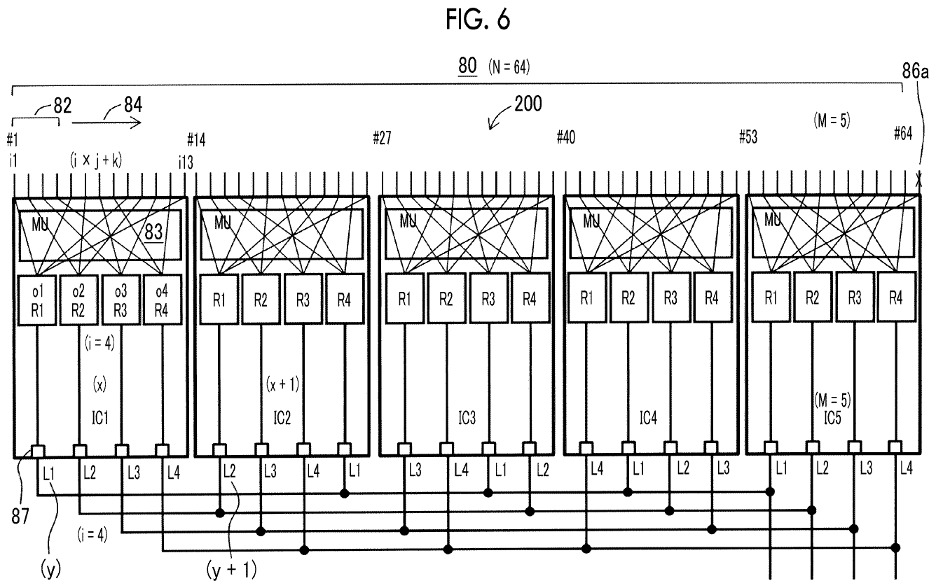

FIG. 6 shows a reception system according to the first example. Specifically, FIG. 6 shows M electronic circuits IC1 to IC5. In the illustrated configuration example, M is five. The electronic circuits IC1 to IC5 are connected to a transducer array 80. The transducer array 80 is configured of N transducers disposed side by side in the electronic scanning direction. In the illustrated configuration example, N is 64. A reference numeral 82 indicates the reception opening located at a specific position. A reception opening 82 is configured of i transducers. In the illustrated configuration example, i is four.

A reference numeral 84 indicates the electronic scanning direction. In FIG. 6, the electronic circuit IC1 located on a leftmost side is an initial (first) electronic circuit, and the electronic circuit IC5 located on a rightmost side is a last (fifth) electronic circuit. This number is a number for convenience, and any electronic circuit may be defined as the first electronic circuit. As will be described below, in the last electronic circuit, the non-use portion that is not connected to the transducer array 80 may be generated. In FIG. 1, the transducer located on the leftmost side among N transducers is a first transducer #1, and the transducer located on the rightmost side is an N-th (64th) transducer.

As already described, each of the electronic circuits IC1 to IC5 includes the multiplexer MU and i receivers R1 to R4. In addition, each of the electronic circuits ICI to IC5 has i internal signal lines provided on an output side of the i receivers R1 to R4 and i connection points (pads) 87 connected to i internal signal lines. The i connection points 87 are connected with i signal lines L1 to L4.

As already described, the multiplexer MU has the (i×j+k) input terminals and the i output terminals. In addition, the multiplexer MU includes a connection path group 83 consisting of the (i×j+k) connection paths connecting the (i×j+k) input terminals and the i output terminals. In FIG. 6, i1 indicates a head input terminal, and i13 indicates a last input terminal. The o1 to o4 represent the i output terminals. The multiplexer MU circularly connects the i signal lines disposed side by side in a predetermined order one-to-one, from the head input terminal to the last input terminal in the (i×j+k) input terminals. The multiplexer MU also functions as a transmission/reception separation circuit.

The five electronic circuits IC1 to IC5 have the same configuration. Input terminals i1 to i13 of the multiplexer MU in the first electronic circuit IC1 are connected to a first transducer group, which is configured by transducers #1 to #13. The input terminals i1 to i13 of the multiplexer MU in the second electronic circuit IC2 are connected to a second transducer group, which is configured by transducers #14 to #26. The input terminals i1 to i13 of the multiplexer MU in the third electronic circuit IC3 are connected to a third transducer group, which is configured by transducers #27 to #39. The input terminals i1 to i13 of the multiplexer MU in the fourth electronic circuit IC4 are connected to a fourth transducer group, which is configured by transducers #40 to #52. The input terminals i1 to i12 of the multiplexer MU in the fifth electronic circuit IC5 are connected to a fifth transducer group, which is configured by transducers #53 to #64. In the fifth electronic circuit IC5, a last input terminal 86a of the multiplexer MU is not used in the illustrated configuration example. In FIG. 6, an X mark is attached to the input terminal. The first multiplexer MU to the fourth multiplexer MU are each the all-input-terminal use type multiplexer (first multiplexer), and the fifth multiplexer MU is the partial-input-terminal non-use type multiplexer (second multiplexer).

The M electronic circuits IC1 to IC5 disposed side by side in the electronic scanning direction have M multiplexers MU disposed side by side in the electronic scanning direction. The wiring circuit 200 is configured by the multiplexers. In order to prevent the overlapping connection in the process of performing the electronic scanning of the reception openings in the electronic scanning direction, in the first example, the electronic circuits IC1 to IC5 are connected to the signal lines L1 to L4 such that the following first connection condition is satisfied.

In a case where an x-th (where x is 1, 2, . . . , and M−1) multiplexer and an (x+1)-th multiplexer adjacent to each other in the electronic scanning direction (typically, in a case where the reception opening is set across the x-th and (x+1)-th multiplexers) are focused on, in a case where a y-th (where y is an integer of 1 or more and i or less) signal line is connected to a last input terminal i13 of the x-th multiplexer, a (y+1)-th (where first in case of y=i) signal line is connected to a head input terminal i1 of the (x+1)-th multiplexer.

Hereinafter, the present disclosure will be specifically described. In the first electronic circuit IC1, the output terminals o1, o2, o3, and o4 (that is, receivers R1, R2, R3, and R4) of the multiplexer MU are connected to the signal lines L1, L2, L3, and L4 (description order in each reference numeral string means connection order). In the second electronic circuit IC2, the output terminals o1, o2, o3, and o4 of the multiplexer MU are connected to the signal lines L2, L3, L4, and L1. In the third electronic circuit IC3, the output terminals o1, o2, o3, and o4 of the multiplexer MU are connected to the signal lines L3, L4, L1, and L2. In the fourth electronic circuit IC4, the output terminals o1, o2, o3, and o4 of the multiplexer MU are connected to the signal lines L4, L1, L2, and L3. In the fifth electronic circuit IC5, the output terminals o1, o2, o3, and o4 of the multiplexer MU are connected to the signal lines L1, L2, L3, and L4.

In the fifth electronic circuit IC5, there are the use portion and the non-use portion disposed side by side in the electronic scanning direction in the input terminal group of the multiplexer MU. Specifically, as described above, the last input terminal 86a is the non-use portion.

With the application of the first connection condition described above, even in a case where the reception opening is set across the two multiplexers MU adjacent to each other in the electronic scanning direction within a range from the first multiplexer MU to the M-th multiplexer MU, the overlapping connection does not occur. Further, in the first example, as described below, even in a case where the reception opening is set across the M-th multiplexer MU and the first multiplexer MU, the overlapping connection is avoided without using a special unit.

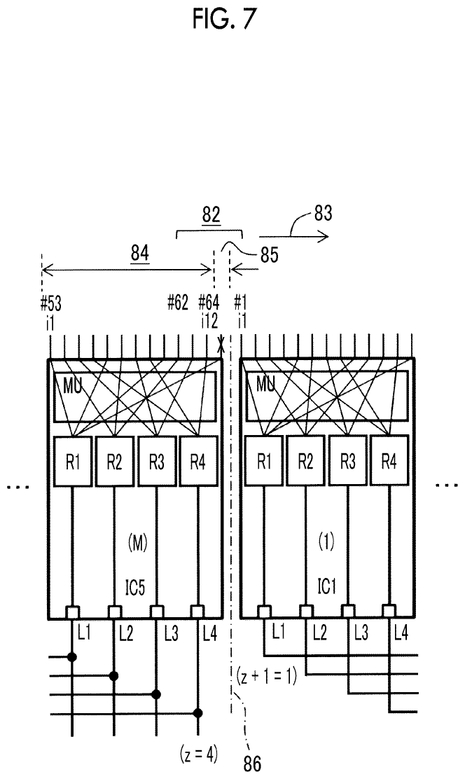

FIG. 7 shows the M-th multiplexer MU and the first multiplexer MU. A reference numeral 86 indicates a gap or a joint between one side and the other side of the FPC substrate. An input terminal row of the M-th multiplexer MU is configured of a use portion 84 and a non-use portion 85 disposed side by side in the electronic scanning direction. The non-use portion 85 is a thirteenth input terminal.

In FIG. 7, the reception opening 82 consists of four transducers from the transducer #62 to the transducer #1. The transducers are connected with tenth, eleventh, and twelfth input terminals in the fifth multiplexer MU and the first input terminal in the first multiplexer MU. The tenth, eleventh, and twelfth input terminals in the fifth multiplexer MU are connected to the signal lines L2, L3, and L4, and the first input terminal in the first multiplexer MU is connected to the signal line L1. In this manner, the overlapping connection does not occur. As indicated by a reference numeral 83, even in a case where the reception opening is shifted by one pitch in the electronic scanning direction, the overlapping connection does not occur. The same applies to subsequent steps.

In general, in a case where the reception opening is set across the M-th multiplexer and the first multiplexer, it is sufficient that the following second connection condition is satisfied in order to avoid the occurrence of the overlapping connection.

In a case where a z-th (where z is an integer of 1 or more and less than i) signal line is connected to a last input terminal of the use portion in the M-th multiplexer, a (z+1)-th (where first in case of z=i) signal line is connected to an initial input terminal in the first multiplexer. In the first example, since the last signal line L4 is connected to the last input terminal i12 of the use portion in the last multiplexer, the second connection condition is naturally satisfied. In a case where the second connection condition is not naturally satisfied, a unit that satisfies the second connection condition is added. This will be described below.

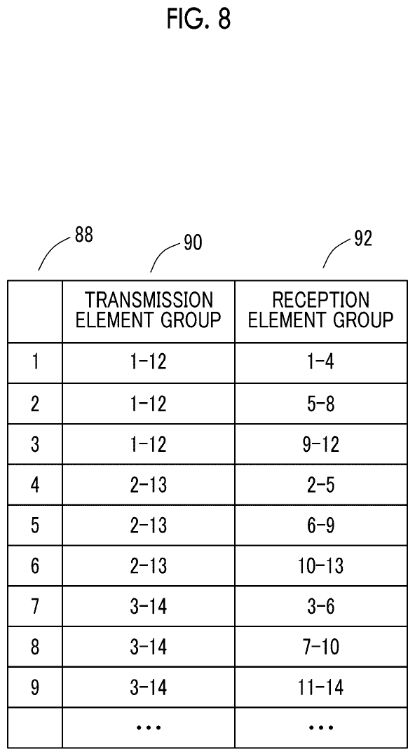

The transmission opening and the reception opening set in the first example will be described with reference to FIG. 8. A reference numeral 88 indicates a transmission/reception number. Reference numeral 90 and reference numeral 92 indicate a transmission element group and a reception element group used in each transmission/reception. The transmission element group is a plurality of transducers constituting the transmission opening. The reception element group is a plurality of transducers constituting the reception opening. In the illustrated example, the transmission opening is configured of 12 transducers, and the reception opening is configured of four transducers. The contents shown in FIG. 8 are exemplary, and various transmission/reception sequences may be employed.

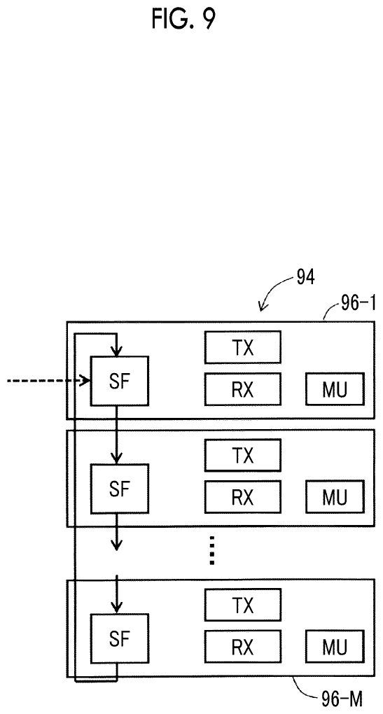

FIG. 9 shows an example of a transfer method of control data. An electronic circuit group 94 is configured of M electronic circuits 96-1 to 96-M. Each of the individual electronic circuits 96-1 to 96-M includes a shift register SF. M shift registers are connected in a ring shape. With an input of a series of pieces of control data to a specific shift register, data necessary for all the shift registers is set. Each of the electronic circuits 96-1 to 96-M operates in accordance with the data.

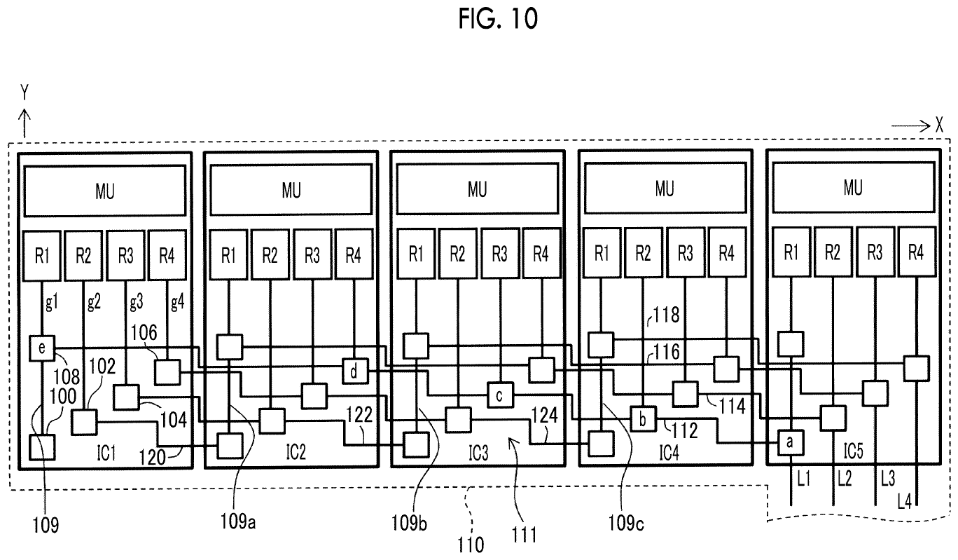

FIG. 10 shows an FPC substrate 110 in the first example. The M electronic circuits IC1 to IC5 are mounted on the FPC substrate 110. The electronic circuits ICI to IC5 have the same configuration. Here, in a case where the electronic circuit IC1 is focused on, the electronic circuit ICI has i internal signal lines gl to g4 connected to the i receivers R1 to R4. In addition, the electronic circuit IC1 has four connection points 100 to 106 provided on the i internal signal lines gl to g4 and a connection point 108 additionally provided on the specific internal signal line g1. Each connection point is a pad or a terminal.

The FPC substrate 110 has a wiring pattern 111. The wiring pattern 111 has a plurality of signal lines 112 to 124. The signal line 112 is used for connecting the signal line L1 to the five specific receivers (refer to a to e). The signal line 114 and the signal line 120 are used for connecting the signal line L2 to the five specific receivers. The signal line 116 and the signal line 122 are used for connecting the signal line L3 to the five specific receivers. The signal line 118 and the signal line 124 are used for connecting the signal line L4 to the five specific receivers.

In the configuration example shown in FIG. 10, the two connection points 100 and 108 are provided on the specific internal signal line g1. A portion 109 between the two connection points 100 and 108 in the specific internal signal line g1 functions as the jumper (short-circuit line) as necessary. That is, two signal lines included in the wiring pattern 111 and separated from each other in a Y direction can be connected by the portion 109 (refer to reference numerals 109a, 109b, and 109c). Accordingly, it is possible to use, as the FPC substrate 110, an FPC substrate having a single wiring layer. Such an FPC substrate is thinner and softer than an FPC substrate having a plurality of wiring layers. Two or three or more connection points are provided for each of the plurality of internal signal lines g1 to g4 as necessary.

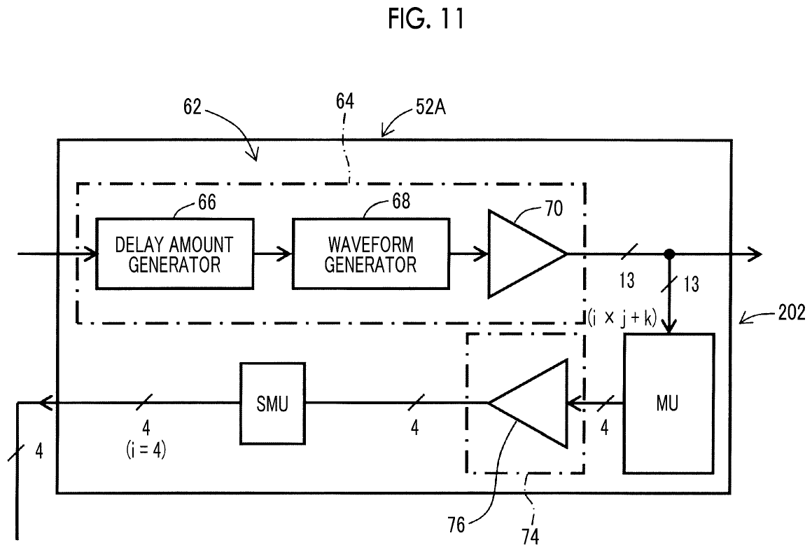

Hereinafter, a second example will be described with reference to FIGS. 11 to 13. FIG. 11 shows an electronic circuit 52A according to the second example. In FIG. 11, the same reference numerals are assigned to the same elements as the elements shown in FIG. 5, and the description thereof will be omitted.

In the electronic circuit 52A, a sub-multiplexer SMU is provided in a post-stage of the reception circuit 74 configured of the i receivers 76. The sub-multiplexer SMU is a unit that prevents the occurrence of the overlapping connection in a specific situation. The sub-multiplexer SMU is configured of, for example, a plurality of switch circuits. In the second example, a wiring circuit 202 is configured of M multiplexers MU and M sub-multiplexers SMU.

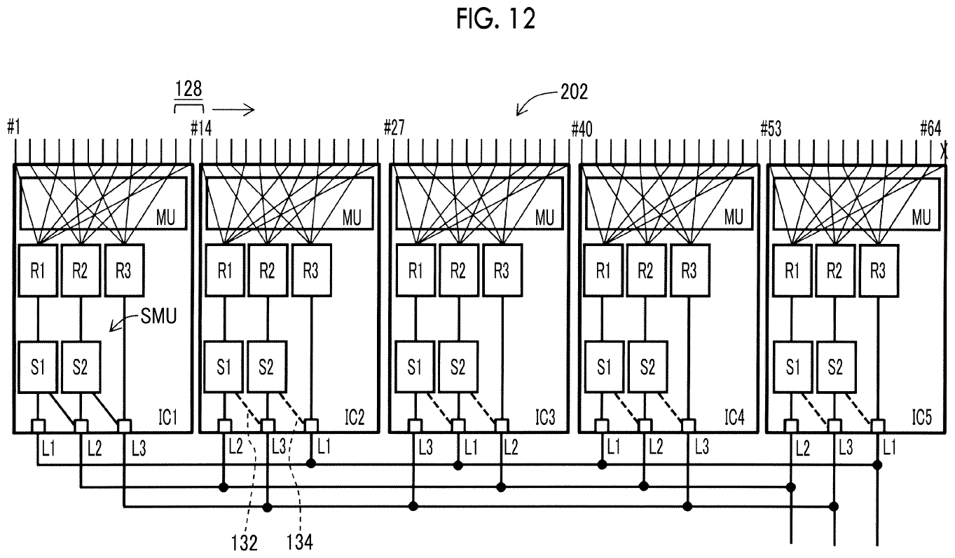

FIG. 12 shows a reception system according to the second example. The reception system includes the M electronic circuits ICI to IC5. The electronic circuits have the same configuration. The transducer array is configured of N transducers. In the illustrated configuration example, M is 5 and N is 64. The number of signal lines i is 3.

Each of the electronic circuits IC1 to IC5 has the multiplexer MU, i receivers R1 to R3, and the sub-multiplexer SMU. The multiplexer MU has the (i×j+k) input terminals and the i output terminals. In addition, the multiplexer MU has the connection path group consisting of the (i×j+k) connection paths connecting the (i×j+k) input terminals and the i output terminals. Here, (i×j+k) is 13 in the illustrated configuration example. That is, i is 3, j is 4,and k corresponding to the remainder is 1. A last input terminal of an M-th multiplexer is not used (refer to X mark). Since the transmission signal is not input to the sub-multiplexer SMU, a circuit having a relatively low breakdown voltage may be used as the sub-multiplexer SMU.

In the illustrated configuration example, the sub-multiplexer SMU is configured of two switch circuits S1 and S2. The switch circuit SI is provided in a post-stage of the receiver R1 and selects any one of the two signal lines. The switch circuit S2 is provided in a post-stage of the receiver R2 and selects any one of the two signal lines.

The signal line connected directly below each of the switch circuits S1 and S2 is a signal line that is selected steadily, and the signal line shown diagonally to the lower right of each of the switch circuits S1 and S2 is a signal line that is selected exceptionally. In practice, only the sub-multiplexer SMU in the first electronic circuit ICI functions, and the sub-multiplexers SMU in the other electronic circuits IC2, IC3, IC4, and IC5 do not function. That is, lines 132 and 134 shown by broken lines are not actually used.

Also in the second example, i signal lines L1 to L4 are connected to the M electronic circuits IC1 to IC5 such that the first connection condition is satisfied. On the premise that all the switch circuits S1 and S2 are in a steady selection state, and in a case where a y-th signal line is connected to a last input terminal of an x-th multiplexer within a range from the first electronic circuit IC1 to the M-th electronic circuit IC5, a (y+1)-th (where first in case of y=i) signal line is connected to a head input terminal of an (x+1)-th multiplexer. Accordingly, the overlapping connection does not occur in the electronic scanning process of the reception opening from the first electronic circuit IC1 to the M-th electronic circuit IC5.

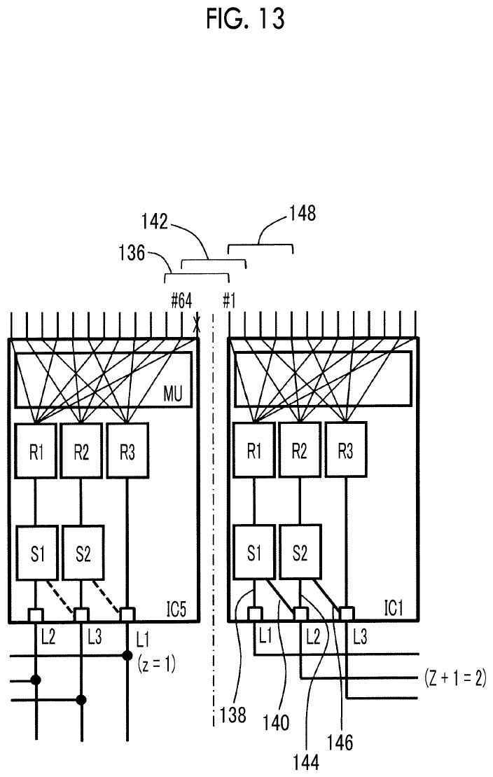

FIG. 13 shows a situation in which a reception opening 136 is set across the M-th multiplexer and the first multiplexer in the second example. In this case, the wiring is performed such that the second connection condition is satisfied.

That is, in a case where a z-th signal line is connected to a last input terminal of the use portion in the M-th multiplexer, a (z+1)-th (where first in case of z=i) signal line is connected to a head input terminal of the first multiplexer.

Specifically, in a case where the reception opening 136 is set, in the first electronic circuit IC1, the switch circuit S1 exceptionally selects, from two output lines 138 and 140, the output line 140. Accordingly, the signal lines L3, L1, and L2 are connected to three transducers constituting the reception opening 136. Subsequently, in a case where a reception opening 142 is set, the switch circuit S1 maintains the selection of the output line 140, and the switch circuit S2 exceptionally selects, from two output lines 144 and 146, the output line 146. Accordingly, the signal lines L1, L2, and L3 are connected to the three transducers constituting the reception opening 136.

As described above, in the second situation in which the reception opening is set across the M-th multiplexer and the first multiplexer, the switch circuits S1 and S2 function as auxiliary, and the overlapping connection is avoided.

At a point in time at which all of the reception openings are within a responsible range of the first multiplexer MU (refer to reference numeral 148), the switch circuit S1 selects the output line 138, and the switch circuit S2 selects the output line 144. That is, an initial wiring state or a steady wiring state is restored.

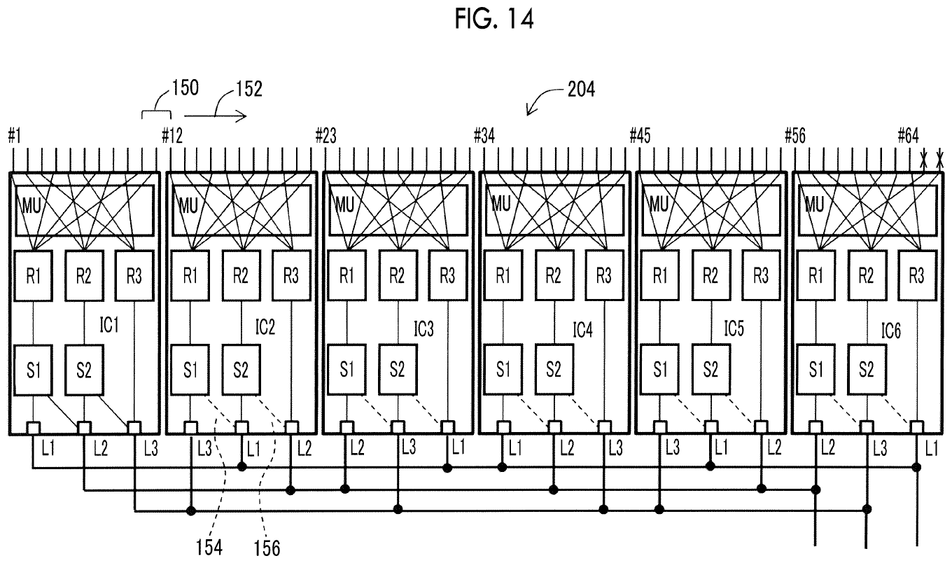

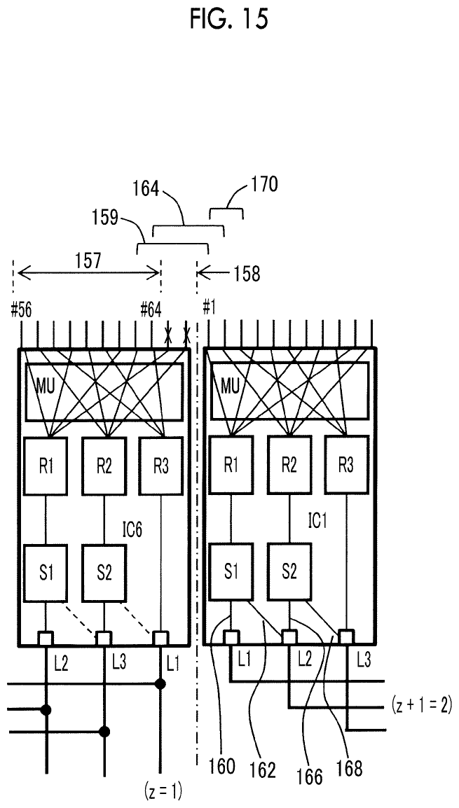

A third example will be described with reference to FIGS. 14 and 15. A configuration according to the third example is basically the same as the configuration according to the second example. However, in the third example, M is 6, N is 64, and i is 3. The number of input terminals (i×j+k) of each multiplexer is 11. Here, j is 3 and k is 2. The input terminal row of the M-th multiplexer includes the use portion and the non-use portion disposed side by side in the electronic scanning direction. The non-use portion is configured of two input terminals (refer to two X marks). In FIG. 14, a first multiplexer MU to a fifth multiplexer MU are each the all-input-terminal use type multiplexer (first multiplexer), and a sixth multiplexer MU is the partial-input-terminal non-use type multiplexer (second multiplexer). It is also conceivable to make a last input terminal of the fifth multiplexer MU and a last input terminal of the sixth multiplexer MU unused.

A reception system according to the third example includes M electronic circuits IC1 to IC6. A wiring circuit 204 is configured of M multiplexers MU and M sub-multiplexers. Each of the individual sub-multiplexers is configured of the switch circuit S1 and the switch circuit S2. Also in the third example, the switch circuit S1 and the switch circuit S2 in the first electronic circuit IC1 function as auxiliary in the second situation. The switch circuits other than the two switch circuits do not operate and steadily select specific signal lines (solid lines). For example, in the second electronic circuit IC2, signal lines (broken lines) 154 and 156 are not selected.

Also in the third example, in the first situation, i signal lines LI to L3 are circularly connected to the M electronic circuits IC1 to IC6 such that the first connection condition is satisfied. That is, on the premise that all the switch circuits SI and S2 are in a steady selection state, in a case where a y-th signal line is connected to a last input terminal of an x-th multiplexer within a range from the first electronic circuit IC1 to the M-th electronic circuit IC5, a (y+1)-th (where first in case of y =i) signal line is connected to a head input terminal of an (x+1)-th multiplexer. Accordingly, the overlapping connection does not occur in the electronic scanning process of the reception opening from the first electronic circuit IC1 to the M-th electronic circuit IC6.

FIG. 15 shows an operation of the third example in the second situation. The input terminal row in the M-th multiplexer is configured of a use portion 157 and a non-use portion 158 disposed side by side in the electronic scanning direction. A reception opening 159 is set across an M-th multiplexer and a first multiplexer. In this case, the wiring is performed such that the second connection condition is satisfied.

That is, in a case where a z-th signal line is connected to a last input terminal of the use portion 157 in the M-th multiplexer, a (z+1)-th (where first in a case of z=i) signal line is connected to a head input terminal of the first multiplexer. The switch circuits S1 and S2 are operated such that the second connection condition is satisfied.

Specifically, a first signal line L1 is connected to the last input terminal of the use portion 157 via a receiver R3. At a point in time of setting the reception opening 159, the switch circuit S1 selects an output line 162. That is, a signal line L2 is selected. Accordingly, three reception signals output from three transducers constituting the reception opening 159 are transmitted to signal lines L3, L1, and L2.

Thereafter, at a point in time of setting the reception opening 164, the switch circuit S1 maintains the selection of the output line 162, and the switch circuit S2 selects an output line 168. That is, the signal line L3 is selected. Accordingly, three reception signals output from three transducers constituting the reception opening 164 are transmitted by using the signal lines L1, L2, and L3.

At a point in time at which all of the reception openings are within a responsible range of the first multiplexer MU (refer to reference numeral 170), the switch circuit S1 selects the signal line 160, and the switch circuit S2 selects a signal line 166. That is, an initial wiring state or a steady wiring state is restored.

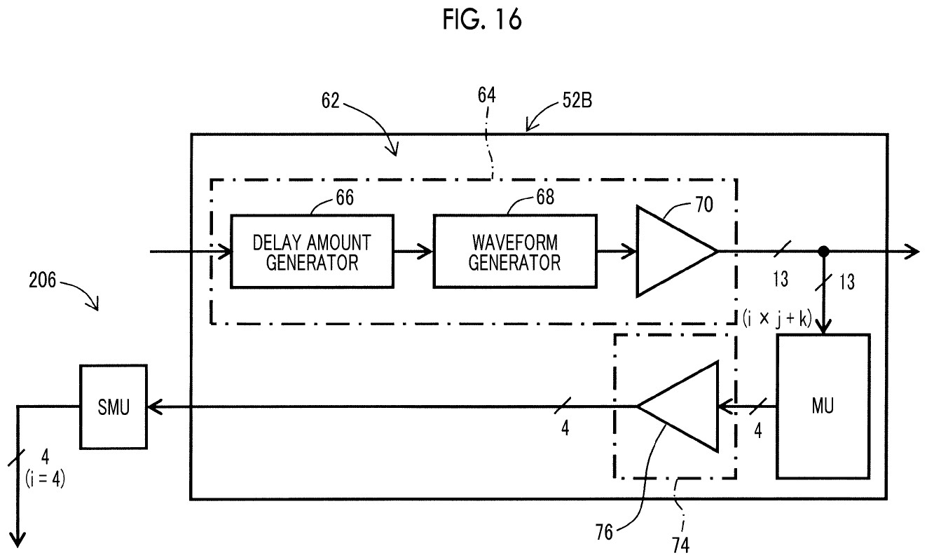

FIG. 16 shows a modification example according to the second example and the third example. In FIG. 16, the same reference numerals are assigned to the same elements as the elements shown in FIG. 11, and the description thereof will be omitted.

An electronic circuit 52B does not have the sub-multiplexer SMU. The sub-multiplexer SMU is added to a post-stage of the first electronic circuit 52B. A wiring circuit 206 is configured of the M multiplexers MU and one sub-multiplexer SMU. According to the present modification example, it is possible to miniaturize each electronic circuit while satisfying the second connection condition. The sub-multiplexer SMU may be mounted on an FPC substrate for wiring.

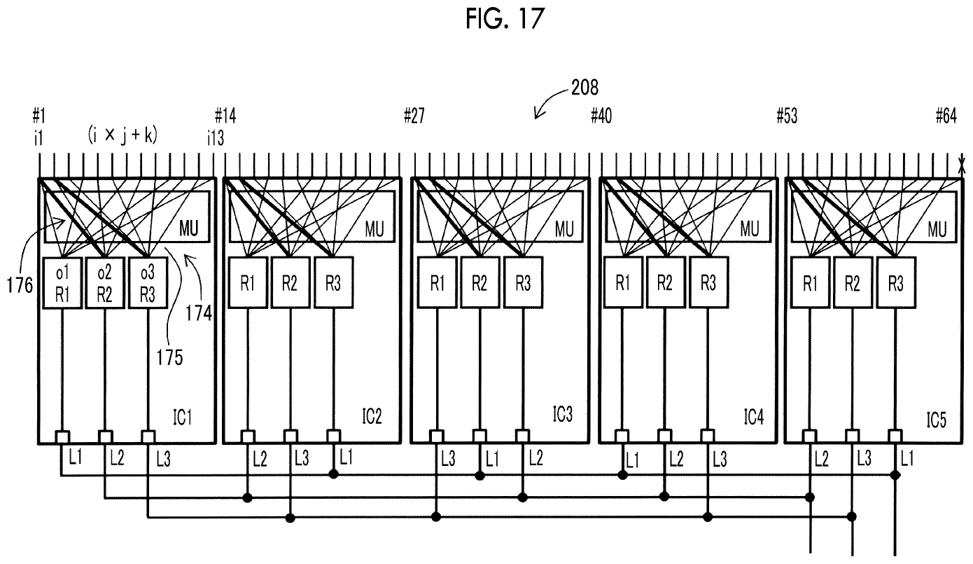

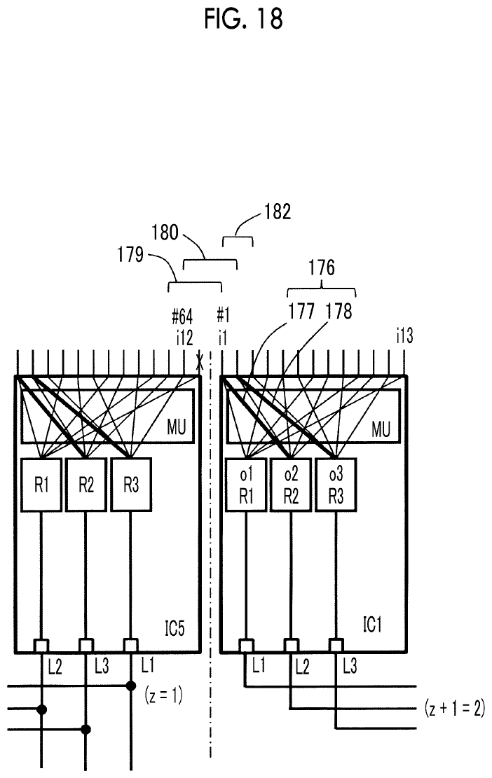

Next, a fourth example will be described with reference to FIGS. 17 and 18. In FIG. 17, a reception system according to the fourth example includes M electronic circuits IC1 to IC5. The M electronic circuits IC1 to IC5 have the same configuration. In the illustrated configuration example, M is 5, N is 64, and i is 3. Each of the individual electronic circuits IC1 to IC5 has a multiplexer MU and i receivers R1 to R3. A last input terminal of an M-th multiplexer is not used (refer to X mark).

Each multiplexer MU has (i×j+k) input terminals i1 to i13 and i output terminals o1 to o3. The (i×j+k) input terminals i1 to i13 are connected to (i×j+k) transducers. The i output terminals are connected to the i signal lines L1 to L3. In the illustrated configuration example, (i×j+k) ) is 13, that is, j is 4 and k is 1.

Each multiplexer MU has a connection path group 174. The connection path group 174 includes a connection path group 175 consisting of (i×j+k) connection paths for circularly connecting the i signal lines L1 to L3 to the (i×j+k) transducers. In addition, the connection path group 174 includes an additional connection path group 176 consisting of (i−1) connection paths (additional connection paths) for avoiding the overlapping connection in the second situation described above. In the illustrated example, the additional connection path group 176 is configured of (i-1) additional connection paths, and specifically, is configured of two additional connection paths (refer to two thick lines).

In the first electronic circuit IC1, in a steady state, a first input terminal is connected to the first signal line L1, and a second input terminal is connected to the second signal line L2. In the second situation described above, in the first electronic circuit IC1, the first input terminal is connected to the second signal line L2, and the second input terminal is connected to the third signal line L3. M-1 additional connection path groups included in a second multiplexer MU to an M-th multiplexer MU do not function. Only the additional connection path group included in the first multiplexer MU functions to avoid the overlapping connection only in the second situation.

Also in the fourth example, the i signal lines L1 to L3 are connected to the M electronic circuits IC1 to IC5 such that the first connection condition described above is satisfied. That is, in a case where a y-th signal line is connected to a last input terminal of an x-th multiplexer, a (y+1)-th (where first in case of y=i) signal line is connected to a head input terminal of an (x+1)-th multiplexer. In that case, a (y+2)-th (where second in case where y=i) signal line is connected to a second input terminal of the (x+1)-th multiplexer. The same applies to other input terminals in the (x+1)-th multiplexer.

FIG. 18 shows an operation of the fourth example in the second situation. A reception opening 179 is set across the M-th electronic circuit IC5 and the first electronic circuit IC1. The first signal line L1 is connected to a last input terminal i12 of a use portion in the M-th multiplexer MU via a receiver R3. That is, y=1. In this case, with an action of the first multiplexer MU (with the use of the additional connection path group 176), the second signal line L2, instead of the first signal line L1, is connected to a head input terminal i1 of the first multiplexer MU (refer to reference numeral 177).

Thereafter, at a point in time at which a reception opening 180 is set, in the first multiplexer MU, the connection of the second signal line L2 to the head input terminal i1 is maintained, and the third signal line L3, instead of the second signal line L2, is connected to the second input terminal (refer to reference numeral 178). At a point in time at which all of the reception openings are within a responsible range of the first multiplexer MU (refer to reference numeral 182), an initial connection state or a steady connection state is restored.

According to the first example, the overlapping connection is naturally avoided in the second situation. In general, in a case where the last input terminal of the use portion in the M-th multiplexer is connected to the i-th signal line, the overlapping connection is naturally avoided. According to the second example, the third example, and the fourth example, since the unit that avoids the overlapping connection is provided in the second situation, the overlapping connection is avoided by using the unit.

What is claimed is:

1. An intracavitary insertion type ultrasound probe comprising:
an annular transducer array;
i (where i is an integer of two or more) signal lines; and
a wiring circuit including a plurality of multiplexers that are disposed side by side in parallel between the transducer array and the i signal lines, wherein each of the multiplexers has (i×j+k) (where j is an integer of one or more and k is an integer of one or more and less than i) input terminals to which a plurality of reception signals from the transducer array are input and i output terminals connected to the i signal lines, and
the wiring circuit connects, in a reception beam scanning process in which a plurality of reception openings are sequentially set to the transducer array, the i signal lines to i transducers constituting each reception opening without overlapping connection.

2. The intracavitary insertion type ultrasound probe according to claim 1,
wherein each of the multiplexers includes (i×j+k) connection paths for circularly connecting the i output terminals one-to-one, from a head input terminal which is a first input terminal to a last input terminal which is (i×j+k)-th input terminal.

3. The intracavitary insertion type ultrasound probe according to claim 2,
wherein the plurality of multiplexers are M (where M is an integer of two or more) multiplexers, and
in a first situation in which the reception opening is set across an x-th (where x is 1, 2, . . . , and M-1) multiplexer and an (x+1)-th multiplexer adjacent to each other in an electronic scanning direction, in a case where a y-th (where y is an integer of one or more and i or less) signal line is connected to a last input terminal of the x-th multiplexer, the wiring circuit connects a (y+1)-th (where first in case of y≈i) signal line to a head input terminal of the (x+1)-th multiplexer.

4. The intracavitary insertion type ultrasound probe according to claim 3,
wherein the (i×j+k) input terminals in an M-th multiplexer include a use portion and a non-use portion disposed side by side in the electronic scanning direction, and
in a second situation in which the reception opening is set across the M-th multiplexer and a first multiplexer, in a case where a z-th (where z is an integer of one or more and i or less) signal line is connected to a last input terminal of the use portion in the M-th multiplexer, the wiring circuit connects a (z+1)-th (where first in case of z=i) signal line to a head input terminal of the first multiplexer.

5. The intracavitary insertion type ultrasound probe according to claim 2,
wherein the plurality of multiplexers include at least one first multiplexer and at least one second multiplexer,
all of the (i×j+k) input terminals are used in the at least one first multiplexer, and
the (i×j+k) input terminals include a use portion and a non-use portion in the at least one second multiplexer.

6. The intracavitary insertion type ultrasound probe according to claim 1, further comprising:
M electronic circuits provided between the transducer array and the i signal lines,
wherein each of the electronic circuits includes the multiplexer, and i receivers provided between the i output terminals of the multiplexer and the i signal lines.

7. The intracavitary insertion type ultrasound probe according to claim 6,
wherein a plurality of the electronic circuits have the same configuration.

8. The intracavitary insertion type ultrasound probe according to claim 7, further comprising:
a circuit board having a wiring pattern for connecting the i signal lines to the plurality of electronic circuits.

9. The intracavitary insertion type ultrasound probe according to claim 8,
wherein each of the electronic circuits has i internal signal lines drawn from the i receivers,
the i internal signal lines are connected to the wiring pattern,
a specific internal signal line among the i internal signal lines has two connection points, and
a portion between the two connection points in the specific internal signal line functions as a jumper connecting two signal lines separated from each other in the wiring pattern.

10. The intracavitary insertion type ultrasound probe according to claim 4,
wherein the wiring circuit includes a sub-multiplexer provided between i output terminals of the first multiplexer and the i signal lines, and
the M-th multiplexer, the first multiplexer, and the sub-multiplexer connect the i signal lines to the i transducers constituting the reception opening without overlapping connection in the second situation.

11. The intracavitary insertion type ultrasound probe according to claim 4,
wherein the wiring circuit includes an additional connection path group consisting of (i −1) additional connection paths added to the first multiplexer, and
the M-th multiplexer and the first multiplexer connect the i signal lines to the i transducers constituting the reception opening without overlapping connection in the second situation.
